(19) United States
(12) Patent Application Publication
(10) Pub. No.: US 2010/0145347 A1
(43) Pub. Date: Jun. 10, 2010

(54) CENTERING JIG FOR RESURFACING THE HEAD OF THE FEMUR AND METHOD

(76) Inventors: Michel Serrault, Flers (FR); Philippe Piriou, Belloy En France (FR); Xavier Collard, Erbisoeul (BE); Philippe Laffargue, La Madeleine (FR); Jean-Paul Forthomme, Saint Symphorien (BE); Henri Migaud, Lille (FR); Yannick Pinoit, Lille (FR); Emmanuel Lizee, Saint Ismier (FR); Alain Tornier, Saint Ismier (FR); Yves-Alain Ratron, Grenoble (FR)

Correspondence Address: FAEGRE & BENSON LLP;PATENT DOCKETING - INTELLECTUAL PROPERTY, 2200 WELLS FARGO CENTER, 90 SOUTH SEVENTH STREET, MINNEAPOLIS, MN, 55402-3901, US

(21) Appl. No.: 12/444,083

(22) PCT Filed: Oct. 1, 2007
(86) PCT No.: PCT/FR07/01601

§ 371 Date: Feb. 24, 2010

Related U.S. Application Data
(60) Provisional application No. 60/852,417, filed on Oct. 18, 2006.

(30) Foreign Application Priority Data
Oct. 2, 2006 (FR) 06 08615

(51) Int. Cl. A61B 17/17

(52) U.S. Cl. 606/89

(57) ABSTRACT

A jig for resurfacing the head of a femur comprises a centering guide defining a centering axis. It further comprises: a support suitable for fitting on a head of the femur and defining an envelope surface forming a portion of a sphere, the centering guide being fastened to the support with the centering axis aligned on a diameter of the sphere; and at least one abutment arm for coming into abutment against a predefined portion of a neck of the femur, the abutment arm being connected to the support and having a distal end that bears against the corresponding predefined portion when the support is fitted on the head of the femur with the centering axis aligned on the central axis of the neck of the femur or with a small amount of valgus.

### CROSS REFERENCE TO RELATED APPLICATION

This patent application is a national stage application of International Application No. PCT/FR2007/001601, filed Oct. 1, 2007, which claims priority to French Patent Application No. 06 08615, filed on Oct. 2, 2006, and also claims the benefit of U.S. Provisional Patent Application No. 60/852,417, filed on Oct. 18, 2006. The entire content of these applications are hereby expressly incorporated by reference.

### TECHNICAL FIELD

The present invention relates to a centering jig for positioning a prosthetic component for resurfacing a head of a femur. In the meaning of the invention, positioning a prosthetic component for resurfacing the head of the femur comprises both preparing the femur and actually implanting the prosthetic component on the head of the femur.

### BACKGROUND

In a conventional manner, a prosthesis for resurfacing a hip includes a component for resurfacing a head of a femur. The size of this component and its positioning are determined at the preoperative stage on the basis of an analysis of one or more X-ray images of the hip. Optimum positioning of the femoral component corresponds to aligning a rod of the component on a central axis of a neck of the femur or with a small amount of valgus relative thereto, while passing, if possible, through a center of the head of the femur. Such positioning of the femoral component enables anatomical criteria to be complied with and guarantees that the implant presents good mechanical behavior. Furthermore, such positioning serves to avoid the neck of the femur being damaged while the femur is being prepared, and in particular while the head of the femur is being trimmed by milling, thereby limiting any risk of subsequent fracture of the neck of the femur.

For positioning a femoral component, it is known to make use of a centering jig for setting an optimum axis for implanting the femoral component relative of the head of the femur, which axis is previously determined on the basis of X-ray analysis. Setting on this optimum implantation axis is essential, since that is what determines the direction in which the head of the femur is drilled and the approach direction for all of the tools used for shaping the femur.

US-A-2005/033290 describes a centering jig including a centering guide of centering axis that is designed to be put into alignment on the optimum axis for implantation as determined during preoperative planning. This alignment is obtained by means of a clamp connected to the centering guide and designed to encompass the neck of the femur. The positioning of the centering guide is determined by adjusting the angle of inclination of the clamp relative to the neck of the femur by aligning a rod on the central axis of the neck of the femur in anterior view. Such a jig is of considerable size, since the alignment rod is offset laterally relative to the centering guide and to the clamp. Furthermore, that centering jig requires alignment of the rod on the central axis of the neck of the femur to be evaluated, which is difficult to do and requires measurement means in order to obtain good accuracy.

### SUMMARY

The present invention seeks more particularly to remedy those drawbacks by proposing a centering jig that presents limited size and that enables its centering axis to be positioned easily and accurately.

To this end, the invention provides a centering jig for positioning a prosthetic component for resurfacing a head of a femur, the jig comprising a centering guide defining a centering axis, and being characterized in that it further comprises:

a support suitable for fitting on the head of the femur and including at least one portion defining an envelope surface forming a portion of a sphere substantially complementary to the head of the femur, the centering guide being fastened to the support on an outside of the envelope surface, with the centering axis aligned on a diameter of the sphere or parallel to a diameter of the sphere; and

at least one abutment arm for coming into abutment against a predefined portion of a neck of the femur, each abutment arm being connected to the support and having a distal end that bears against a corresponding predefined portion of the neck of the femur when the support is fitted on the head of the femur with the centering axis aligned on the central axis of the neck of the femur or with a small amount of valgus relative to the axis.

According to other characteristics of the centering jig that are advantageous, taken in isolation or in any technically feasible combination:

at least one of the abutment arms is an arm for coming into abutment against an internal portion or an external portion of the neck of the femur;

each abutment arm comprises first and second segments that are substantially perpendicular to each other, the first segment being fastened to the support and extending parallel to the centering axis, while the second segment has the distal end for bearing against the corresponding predefined portion of the neck of the femur;

the centering guide is releasably fastened to the support;

for each predefined portion of the neck of the femur, the centering jig includes a set of abutment arms of different dimensions and is suitable for being fastened in a selective manner to the support;

for each predefined portion of the neck of the femur, the centering jig includes a single abutment arm suitable for being fastened to the support in an adjustable manner to slide along a direction that is substantially parallel and/or a direction that is substantially perpendicular to the centering axis;

the centering jig includes at least one other abutment arm that is an arm for coming into abutment against the anterior portion or the posterior portion of the neck of the femur;

the centering jig includes a fastener pin for fastening the centering guide on the head of the femur, the pin comprising a tubular body provided with distal teeth and suitable for being engaged around the centering guide;

the centering guide is a circular-section tube of central axis forming the centering axis, the centering guide being pierced by slots for monitoring the extent to which a drill bit inserted inside the centering guide has advanced; and

the centering jig includes a device for verifying the position of the centering axis, the device comprising a tubular body suitable for engaging around the centering guide and a rod suitable for feeling the distal portion of the head of the femur around its periphery.

The invention also provides a method of positioning a prosthetic component for resurfacing the head of a femur, the method making use of a centering jig as defined above, and comprising steps of:

covering the head of the femur with the support; and

tilting the support on the head of the femur until the distal end of each abutment arm comes to bear against the corresponding predefined portion of the neck of the femur.

Such a positioning method may include subsequent additional steps of:

verifying the position of the centering axis by causing the rod of the verification device to turn about the junction between the head and the neck of the femur;

preventing the centering jig from moving on the head of the femur by impacting the distal teeth of the fastener pin in the head of the femur; and

drilling the head of the femur along the centering axis by means of a bit inserted inside the centering guide.

### BRIEF DESCRIPTION OF THE DRAWINGS

The characteristics and advantages of the invention appear in the following description of an embodiment of a centering jig of the invention, given purely by way of example and made with reference to the accompanying drawings, in which:

FIG. 1 is an exploded perspective view of a centering jig in accordance with the invention;

FIG. 2 is a section on plane II of FIG. 1;

FIG. 3 is a perspective view showing the FIG. 1 centering jig from the same angle during a first step of centering an optimum implantation axis for positioning a prosthetic component for resurfacing the head of the femur; and

FIG. 4 is a perspective view of the FIG. 1 centering jig from the same angle, during a second centering step.

### DETAILED DESCRIPTION

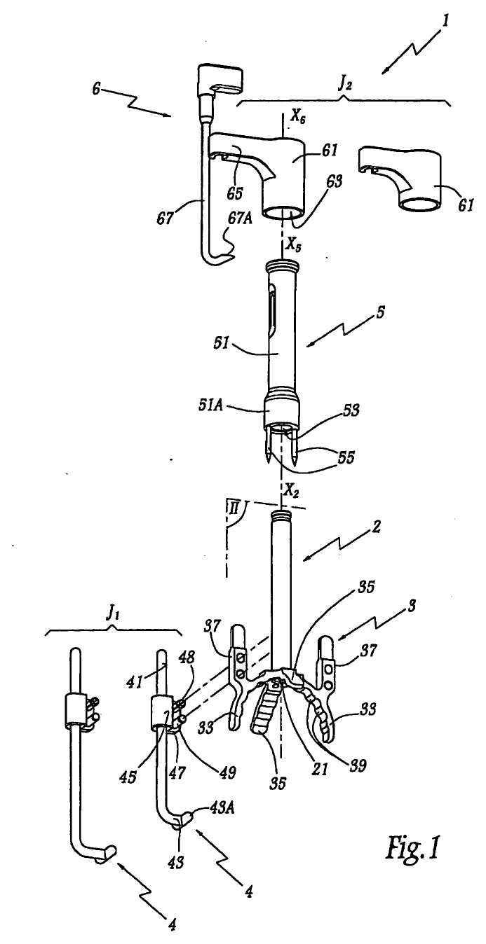

A centering jig 1 shown exploded in FIG. 1 comprises a centering guide 2 in the form of a circular section tube having a central bore 21. A central axis X₂ of the bore 21 constitutes the centering axis of the jig 1. The centering guide 2 is suitable for receiving a drill bit (not shown) in its bore 21, with the central axis of the bit being substantially aligned on the centering axis X₂. The centering guide 2 then constitutes a drilling guide for the bit.

The centering guide 2 is designed to be positioned facing a head 71 of a femur 7 that is to have a femur resurfacing component implanted thereon, and the centering axis X₂ is designed to be aligned on an optimum axis for implanting the femoral component. This alignment is obtained by using the jig 1 having a configuration that is adapted on the basis of parameters, e.g. parameters that are determined during preoperative planning or statistically. Once the centering axis X₂ has been positioned in this way, a bit inserted in the bore 21 can be used to drill the head 71 of the femur 7 on the optimum implantation axis.

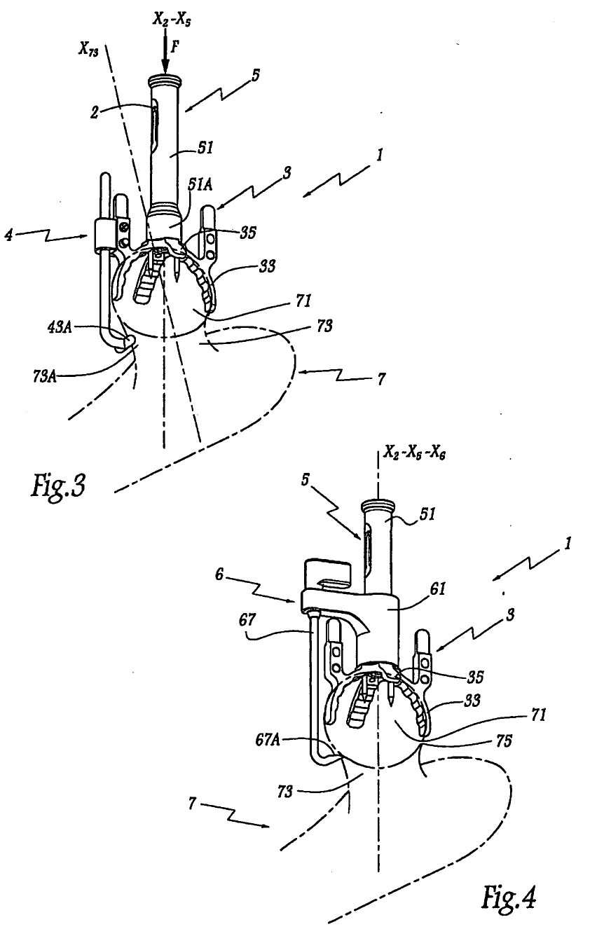

The centering guide 2 is rigidly secured to a support 3 made up of a plurality of portions 31, 33, and 35, that together define an envelope surface E₃ of hemispherical shape that is substantially complementary to the head 71 of the femur 7. In particular, in the example shown, the centering guide 2 is fastened on the support 3 with the centering axis X₂ passing through the top of the hemisphere formed by the envelope surface E₃ and forming a diameter of the hemisphere. The support 3 is suitable for fitting over the top portion of the head 71 of the femur 7, as can be seen in FIGS. 3 and 4. When the support 3 is in position on the head 71 of the femur 7, the centering axis X₂ passes substantially through the center of the head 71 of the femur 7.

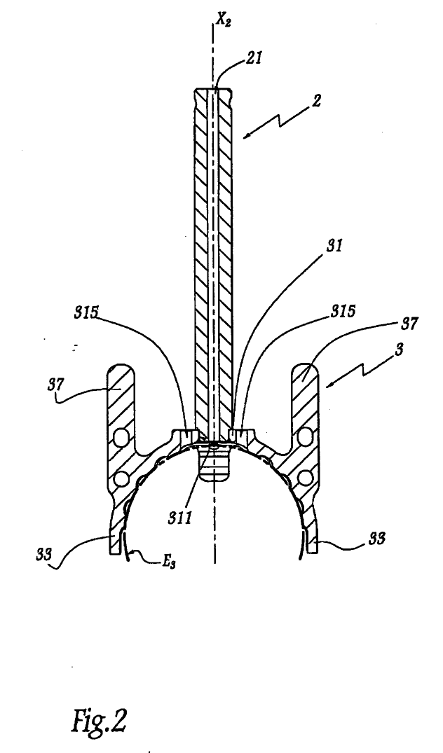

The support 3 has a top portion 31 pierced by a central orifice 311 that is positioned to correspond with the bore 21 of the centering guide 2, as can be seen in FIG. 2. Four tabs 33 and 35 extend from the top portion 31 and are regularly distributed around the top portion 31. Two tabs 33 placed facing each other serve to bear respectively against internal and external portions of the head 71 of the femur 7. The other two tabs 35, likewise placed facing each other, are for bearing respectively against anterior and posterior portions of the head 71 of the femur. On its concave surface, each tab 33 or 35 includes ridges 39 for engaging the head 71 of the femur.

In the example shown, the two tabs 33 are of the same length, this length also being the length of the tab 35 that is to bear against the posterior portion of the head 71 of the femur. The tab 35 that is to bear against the anterior portion of the head 71 of the femur is shorter than the other tabs. Such a configuration of the tabs 33 and 35 makes it possible to obtain good stability for the support 3 when fitted on the head 71 of the femur.

In a variant, the two tabs 35 could both be shorter than the tabs 33. The support 3 is then less stable, but more compact, thereby reducing the overall size of the jig 1.

The jig 1 also has an abutment arm 4 releasably fastened to the support 3. In the example shown, a distal end 43A of the abutment arm 4 is designed to bear against the internal portion 73A of the neck 73 of the femur 7, or “thigh spur”. The support 3 is provided with two posts 37 for fastening the abutment arm 4, each disposed on the convex surface of one of the tabs 33 of the support 3 and symmetrical to each other about the centering axis X₂. Thus, the abutment arm 4 is adapted to be fastened on the support 3 via one or the other of the posts 37, depending on whether the femur 7 is the right femur or the left femur of the patient. In the variant where both tabs 35 are of the same length, the symmetry of the support 3 means that it is possible to provide only a single post 37 for fastening the abutment arm 4.

The abutment arm 4 comprises a proximal segment 41 and a distal segment 43, which segments are substantially perpendicular to each other. The proximal segment 41 is provided with a sleeve 45 that is fastened rigidly to the segment 41 and that is extended by a plate 47 for fastening to a post 37. In the example shown, the plate 47 includes a spring clip 48 suitable for locking in a corresponding orifice provided in each post 37. In a variant, each post 37 may have a tapped orifice suitable for receiving a screw passing through a corresponding orifice in the plate 47. In the example shown, the abutment arm 4 is designed to be fastened on the support 3 with its proximal segment 41 substantially parallel to the centering axis X₂. To guarantee this positioning, the plate 47 is provided with a peg 48 suitable for being inserted in a corresponding orifice in each post 37. When the abutment arm 4 is fastened on the support 3, the distal segment 43 that includes the distal end 43A extends towards the centering axis X₂, substantially perpendicularly to the axis.

The dimensions of the abutment arms 4 are determined on the basis of parameters, e.g. parameters that are obtained during preoperative planning or statistically, such that, when the support 3 is fitted on the head 71 of the femur with the distal end 43A of the abutment arm 4 bearing against the internal portion 73A of the neck 73 of the femur, the centering axis X₂ is in alignment on an optimum axis for implanting the femoral component. The dimensions of the abutment arm 4 depend on the morphology of the femur 7. In the embodiment shown, the jig 1 is advantageously provided with a set J₁ of abutment arms 4 of different dimensions, adapted to be fastened selectively on the support 3. In a variant, a jig in accordance with the invention may have a single abutment arm 4 adapted to be fastened in an adjustable manner on the support 3. In particular, such adjustment may be implemented by causing the abutment arm 4 to slide in a first direction substantially parallel to the centering axis X₂, e.g. by the segment 41 sliding relative to the sleeve 45, and also by causing it to slide in a second direction that is substantially perpendicular to the centering axis X₂.

As can be seen in FIG. 1, the jig 1 also includes a fastener pin 5 comprising a body 51 of circular-section tubular shape that is provided at its distal end 51 A with impact teeth 55. The pin 5 has a central bore 53 centered on an axis X₅ and substantially complementary to the outside dimensions of the centering guide 2. Thus, the pin 5 can be engaged around the centering guide 2 with the axes X₂ and X₅ coinciding. The distal portion 51A of the body 51 is designed to bear against the top portion 31 of the support 3. Orifices 315 are also provided in the top portion 31 in order to pass the distal teeth 55 of the pin 5.

Finally, the jig 1 includes a satellite 6 for verifying the position of the centering axis X₂ relative to the head 71 of the femur. The satellite 6 comprises a body 61 of circular-section tubular shape, provided with a central bore 63 of axis X₆. The bore 63 is complementary to the outside dimensions of the body 51 of the pin 5. Thus, the body 61 of the satellite 6 is suitable for being engaged around the body 51 of the pin 5, with the axes X₅ and X₆ then coinciding. When the centering guide 2, the pin 5, and the satellite 6 are all three engaged one in another, as shown in FIG. 4, the three axes X₂, X₅, and X₆ coincide, and the body 61 of the satellite 6 comes to bear against the proximal ends of the tabs 33 and 35 of the support 3.

The satellite 6 is provided with a rod 67 having a distal end 67A that is suitable for feeling the distal portion of the head of the femur that is to be prepared, in particular by being trimmed by milling, so that the femoral surfacing component can be implanted. The rod 67 is connected to the body 61 via a lateral branch 65 of the tubular body 61. When it is in the position shown in FIG. 4, the body 61 is designed to be capable of being turned about the coinciding axes X₂, X₅, and X₆. Thus, the distal end 67A of the rod 67 is suitable for feeling the head 71 of the femur all around its periphery.

In known manner, the dimensions of the satellite 6 are adjusted at the preoperative stage so as to simulate the size selected for the femoral resurfacing component. In the example shown, the jig 1 is provided with a set J₂ of satellite bodies 61 of different dimensions, suitable for being engaged symmetrically around the pin 5 and the centering guide 2, whereas there is only one rod 67 and it is suitable for co-operating with each of the bodies 61 in the set J₂ of satellite bodies.

The centering jig 1 is used as follows:

Initially, the abutment arm 4 of the set J₁ having the appropriate dimensions is fastened on the support 3 via the appropriate fastener post 37. The pin 5 is then engaged around the centering guide 2, with its impact teeth 55 offset away from the orifices 315 through the portion 31 of the support 3, so that they do not project beyond the concave surface of the portion 31. In this configuration, the jig 1 is suitable for being moved freely over the head 71 of the femur.

While the jig 1 is in the above-described configuration, it is positioned relative to the femur 7. More specifically, the head 71 of the femur is covered by the support 3. The centering axis X₂ then passes substantially through the center of the head 71 of the femur. Thereafter, the support 3 is tilted on the head 71 of the femur until the distal end 43A of the abutment arm 4 comes to bear against the internal portion 73A of the neck 73 of the femur. In this position, as shown in FIG. 3, the centering axis X₂ is substantially in alignment on an optimum axis for implanting the femoral resurfacing component, i.e. presenting a small amount of valgus relative to the central axis X₇₃ of the neck 73 of the femur, and passing through the center of the head 71 of the femur.

The jig 1 is then prevented from moving relative to the head 71 of the femur so that the centering axis X₂ is held in alignment on the optimum axis for implantation. For this purpose, the body 51 of the fastener pin 5 is turned relative to the centering guide 2 and to the support 3 about the coinciding axes X₂ and X₅ so as to insert the distal teeth 55 of the pin 5 into the orifices 315 through the portion 31. A thrust force F is then exerted on the proximal end of the body 51 of the pin 5 so as to impact the teeth 55 into the top portion of the head 71 of the femur, as shown in FIG. 3.

Thereafter, the position of the centering axis X₂ is verified by means of the verification satellite 6, so as to ensure that the femoral resurfacing component will subsequently be properly fitted on the head 71 of the femur. For this purpose, the abutment arm 4 is separated from the support 3, and the body 61 of the satellite 6 is engaged around the fastener pin 5. This puts the jig 1 into the configuration shown in FIG. 4. In this configuration, the body 61 of the satellite 6 is caused to turn about the coinciding axes X₂, X₅, and X₆ so that the distal end 67A of the rod 67 turns about the junction 75 between the head 71 and the neck 73 of the femur. When the centering axis X₂ is correctly positioned, the body 61 and the rod 67 can be rotated without being blocked. If such rotation is not possible, then there is a risk of the neck 73 of the femur being damaged during subsequent steps of preparing the femur 7. It is then necessary to consider repositioning the centering axis X₂.

Once the verification satellite 6 has been used to verify that the femoral resurfacing component will be properly fitted on the head 71 of the femur, a drill bit (not shown in the figures) is inserted in the central bore 21 of the centering guide 2 and it is rotated so as to drill the head 71 of the femur along the centering axis X₂.

Once drilling has been completed, the centering jig 1 is removed from the head 71 of the femur by disassembling its component elements. The subsequent steps of shaping the femur 7 and of implanting the femoral resurfacing component are well known to a person skilled in the art, and they are performed in alignment with the hole drilled by means of the centering jig 1 in accordance with the invention.

As can be seen from the above-described steps, the centering jig 1 of the invention enables the centering axis X₂ to be positioned easily and accurately on an optimum axis for implantation, i.e. in alignment with the central axis X₇₃ of the neck 73 of the femur, or presenting a small amount of valgus relative to the axis, and passing through the center of the head 71 of the femur. The hemispherical shape of the support 3 makes it easy to find the center of the head 71 of the femur. The abutment arm 4 serves to position the centering axis X₂ automatically on an optimum axis for implantation, i.e. in alignment with, or presenting a small amount of valgus relative to, the central axis X₇₃ of the neck 73 of the femur. In addition, this optimum positioning of the centering axis X₂ can be held in reliable manner by impacting the distal teeth 55 of the fastener pin 5 into the head 71 of the femur. Finally, the verification satellite 6 makes it possible to verify systematically that the femoral resurfacing component will be properly fitted on the head 71 of the femur 7 prior to drilling the femur 7.

Furthermore, such a centering jig 1 is compact, which is most advantageous in the context of a surgical operation for resurfacing the hip. Finally, such a centering jig 1 can be adapted to femurs of various morphologies by having the set J₁ of abutment arms 4 of different dimensions, or in a variant by having a single abutment arm 4 that can be fastened in an adjustable manner relative to the support 3.

The invention is not limited to the example described and shown. In particular, the abutment arm 4 may be designed to bear against the external portion of the neck 73 of the femur instead of the internal portion 73A of the neck 73. Furthermore, a centering jig in accordance with the invention may have two abutment arms 4 designed to bear respectively against an internal or external portion of the neck 73 of the femur and against an anterior or posterior portion of the neck 73 of the femur. The combination of such abutment arms 4 that are substantially perpendicular to each other enables the centering axis X₂ to be positioned both in the frontal plane and in the sagittal plane.

In another variant of the invention that is not shown, the centering jig 1 may be modular, the centering guide 2 then being releasably fastened to the support 3 instead of being integral therewith as in the example shown. This variant enables the jig to be disassembled and removed easily after the head 71 of the femur has been drilled.

Furthermore, the centering guide 2 may be provided with slots (not shown) for observing progress of a bit for drilling the head 71 of the femur 7. In particular, the drill bit may be marked in such a manner that when it coincides with the slots in the centering guide 2, that indicates that the bit has been drilled to an optimum depth.

Finally, the centering guide 2 may be positioned on the support 3 via an eccentric part, such that the centering axis X₂ is parallel to a diameter of the hemisphere formed by the support 3. Such a configuration makes it possible to compensate for a possible offset of the head 71 of the femur 7 relative to the central axis X₇₃ of the neck 73 of the femur 7.

1. A centering jig for positioning a prosthetic component for resurfacing a head of a femur, the jig comprising a centering guide defining a centering axis, and being characterized in that it further comprises:
a support suitable for fitting on the head of the femur and including at least one portion defining an envelope surface forming a portion of a sphere substantially complementary to the head of the femur, the centering guide being fastened to the support on the outside of the envelope surface, with the centering axis aligned on a diameter of the sphere or parallel to a diameter of the sphere; and
at least one abutment arm for coming into abutment against a predefined portion of a neck of the femur, each abutment arm being connected to the support and having a distal end that bears against a corresponding predefined portion of the neck of the femur when the support is fitted on the head of the femur with the centering axis aligned on the central axis of the neck of the femur or with a small amount of valgus relative to the axis.

2. A centering jig according to claim 1, wherein at least one of the abutment arms is an arm for coming into abutment against an internal portion or an external portion of the neck of the femur.

3. A centering jig according to claim 1, wherein each abutment arm comprises first and second segments that are substantially perpendicular to each other, the first segment being fastened to the support and extending parallel to the centering axis, the second segment including the distal end for bearing against a corresponding predefined portion of the neck of the femur.

4. A centering jig according to claim 1, wherein the centering guide is releasably fastened to the support.

5. A centering jig according to claim 1, wherein each predefined portion of the neck of the femur includes a set of abutment arms of different dimensions, each being suitable for being fastened in selective manner to the support.

6. A centering jig according to claim 1, wherein each predefined portion of the neck of the femur includes a single abutment arm suitable for being fastened to the support in an adjustable manner to slide along a direction that is substantially parallel and/or a direction that is substantially perpendicular to the centering axis.

7. A centering jig according to claim 1, wherein the centering jig includes at least one other abutment arm that is an arm for coming into abutment against an anterior portion or an posterior portion of the neck of the femur.

8. A centering jig according to claim 1, wherein the centering jig includes a fastener pin for fastening the centering guide on the head of the femur, the pin comprising a tubular body provided with distal teeth and suitable for being engaged around the centering guide.

9. A centering jig according to claim 1, wherein the centering guide is a circular-section tube of the central axis forming the centering axis, the centering guide having slots for monitoring the extent to which a drill bit inserted inside the centering guide has advanced.

10. A centering jig according to claim 1, wherein the centering jig includes a device for verifying a position of the centering axis, the device comprising a tubular body suitable for engaging around the centering guide and a rod suitable for feeling the distal portion of the head of the femur around its periphery.

11. A method of positioning a prosthetic component for resurfacing a head of a femur, the method making use of a centering jig according to claim 8, wherein the method comprises:
covering the head of the femur with the support; and
tilting the support on the head of the femur until the distal end of each abutment arm comes to bear against the corresponding predefined portion of the neck of the femur.

12. A positioning method according to claim 11, wherein the method further comprises:
verifying the position of the centering axis by causing the rod of the verification device to turn about the junction between the head and the neck of the femur;
preventing the centering jig from moving on the head of the femur by impacting the distal teeth of the fastener pin in the head of the femur; and
drilling the head of the femur along the centering axis by means of a bit inserted inside the centering guide.

13. A method of positioning a prosthetic component for resurfacing a head of a femur, the method making use of a centering jig according to claim 1, wherein the method comprises:
covering the head of the femur with the support; and
tilting the support on the head of the femur until the distal end of each abutment arm comes to bear against the corresponding predefined portion of the neck of the femur.
